(19) United States
(12) Patent Application Publication
(10) Pub. No.: US 2007/0238174 A1
(43) Pub. Date: Oct. 11, 2007

(54) Substrates

(76) Inventors: Brian G. Cousins, Litherland (GB); Michael Joseph Garvey, Wirral (GB); John Fink, Liverpool (GB); Rachel Lucinda Williams, Little Neston (GB); Patrick Joseph Doherty, Crosby (GB)

Correspondence Address: WADDEY & PATTERSON, P.C., 1600 DIVISION STREET, SUITE 500, NASHVILLE, TN, 37203, US

(21) Appl. No.: 11/708,660

(22) Filed: Feb. 20, 2007

Related U.S. Application Data
(63) Continuation-in-part of application No. 10/480,780, filed on Jun. 9, 2004.

(51) Int. Cl. C12N 1/36; C12N 5/00; C12N 5/02; C12N 5/04; C12N 5/06

(52) U.S. Cl. 435/375; 435/245

(57) ABSTRACT

The present invention relates to novel substrates, to methods of making them and to uses therefor. The substrates of the invention comprise a base portion and a surface layer covering at least part of the base portion, with a binding layer provided therebetween. The surface layer provides, on at least a part of the substrate, topographical features having at least one nano scale dimension. These topographical features are adapted to inhibit cell or tissue growth thereon and/or therebetween.

### CROSS-REFERENCES TO RELATED APPLICATIONS

This application is a Continuation-In-Part application which claims benefit of co-pending U.S. patent application Ser. No. 10/480,780 filed Jun. 11, 2002, entitled “Substrates”, which was a National Phase filing of International Application No. PCT/GB2002/002652 filed Jun. 11, 2002, entitled “Substrates”, both of which are hereby incorporated by reference.

A portion of the disclosure of this patent document contains material that is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND OF THE INVENTION

The present invention relates to novel substrates, to methods of making them and to uses therefor. In particular the invention concerns such materials, methods and uses for the inhibition of cell growth.

The behavior of cells are influenced by their external environment, both chemical and physical. Understanding interactions which take place between a cell and its substrate is important in connection with such fields as medical implants and prostheses, tissue engineering and pharmaceutical development. One substrate characteristic which has been shown to influence cellular behavior is topography and synthetic structured surfaces have been used to investigate this influence. A review of such investigations may be found in Biomaterials (1999), 20, 573-588. In vivo studies are reported in Biomaterials (1996), 17, 2087-2095.

The modification of surface topography for the control of cellular response is an important area of research in medical engineering that targets several potential end uses, particularly relating to the biocompatibility of materials used in medical and dental devices and implants and in the preparation of materials, such as hygienic surfaces, which deter cell growth thereon. In this area, it is required to control the interfacial reactions that mitigate the appropriate response for a specific application. It is known that the interfacial reactions are influenced by the surface properties of the substrate in terms of the surface chemistry, energy and topography. Of the latter, current research is focused on etching techniques to create the desired topography. Experimental Cell Research (1996), 223, 426-435, discusses the production of micro fabricated grooves and steps by means of dry etching a silica substrata with a reactive ion etching unit. U.S. Pat. No. 4,832,759 also discusses the generation of a plurality of surface discontinuities by means of ion beam etching. Many prior art studies have used photolithographic techniques to engineer surface features with controlled morphology for the study of cell behavior thereon. Other techniques include glancing angle deposition, laser ablation, laser deposition, replica molding of x-ray lithography masters, imprint lithography, micro contact printing and etching and ink-jet printing. For example, Canadian Pat No. 2,323,719 discusses the production of structural elevations by the LIGA lithographic process which incorporates x-ray lithography, electrodeposition and molding. Canadian Pat No. 2,302,118 discloses microstructured surfaces produced mechanically or lithographically. DE-A-19818956 discloses materials with a micro-roughened, bacteria-repellant surface. WO-A-97/12966 discloses methods for producing thin colloidal silica films on substrates for growing cells in culture by spin coating or spraying the substrate with colloidal silica, or dipping the substrate in a colloidal silica solution.

Cell-substrate interactions in the natural environment are influenced by the surface topography of the substrate, the topographical features of which are represented at the nanoscale level. Some of the above-mentioned techniques and prior art disclosures for engineering synthetic surface features are capable of generating topographical features at the nanoscale level but none has so far offered a quick and convenient means to inhibit cell growth at this level. Furthermore, the suitability for commercial application of many of the known substrates is limited. One particular problem with some prior art substrates is the tendency of the micro-structured surface layer to crack or peel.

### BRIEF SUMMARY OF THE INVENTION

It is an object of the present invention to provide a substrate which can be used to inhibit cell growth at the nanoscale level. It is a further object of the invention to provide such a substrate which is sufficiently robust to be useful in commercial applications. Another object of the invention is to provide a substrate which can be manufactured reproducibly, conveniently and without excessive expense.

According to the present invention there is provided a cell growth inhibiting substrate comprising:

a base portion;

a surface layer covering at least part of the base portion, the surface layer providing the substrate with topographical features having at least one nanoscale dimension of from about 1 to about 200 nm; and

a binding layer between the base portion and the surface layer for binding the surface layer in place.

The substrate has been shown to inhibit the growth of a number of different cell types, ranging from mammalian cells, fungal cells (including yeast) and bacterial cells. The substrate of the invention therefore provides a novel material which may find application in a wide variety of circumstances. For example, substrates according to the invention may be used to provide medical implants whose surfaces discourage cell growth. Hygienic surfaces, for use in hospitals, restaurants, kitchens, bathrooms etc. may be formed from substrates according to the invention. Furthermore, existing surfaces may be modified to form the substrates of the present invention. Prior art substrates having nanoscale topography have tended to be unsuitable for many commercial applications as they are not sufficiently robust to withstand exposure to the conditions of such applications. Many prior art substrates have nanoscale topography which cracks, flakes or peels over time, rendering the substrate unsuitable for most applications other than short term academic study.

The term, “inhibit”, “inhibition”, or “inhibiting” in relation to cell growth is defined herein as limiting or preventing cellular proliferation.

In the context of this document, “nanoscale” is used to refer to topographical features having at least one dimension which is measurable at the nanometre level, for example a feature which measures from about 1 to about 200 nm, preferably from about 1 to about 150 nm, even more preferably from about 1 to about 100 nm, and most preferably less than about 50 nm in at least one dimension.

The topographical features may form a random array on the surface layer of the substrate. Such an array may comprise, for example, an agglomeration of peaks and troughs, preferably having substantially the same or similar dimensions and physical characteristics.

Alternatively, the topographical features may form an ordered array on the surface layer of the substrate. A combination of random and ordered arrays may be used also. Whatever form of array adopted by the topographical features, the substrate of the invention preferably comprises nanoscale topographical features separated from other nearest neighbor similar nanoscale topographical features by distances of up to about 1000 nm. For example, when the array comprises individual peaks and troughs, each peak in the array may be separated from its nearest neighbor peaks by distances of up to about 500 nm, preferably no more than about 200 nm. Where the array comprises a series of longitudinal ridges, each ridge in the array may be separated from its nearest neighbor by distances of up to about 1000 nm, preferably no more than about 500 nm.

Preferably the base portion and the surface layer are of different materials and preferably the material of the binding layer is different from one or both of the materials of the base portion and the surface layer. The binding layer may comprise a single layer comprising one or more materials, provided that the binding layer comprises at least one material capable of binding to the base portion and at least one material capable of binding to the surface layer. The same material of the binding layer may bind to both the base portion and to the surface layer, in which case the binding layer may comprise a single material. However, the binding layer may comprise a plurality of materials and may be a composite layer. Thus, for example the binding layer may comprise two layers containing, respectively, a first material and a second material. The first material may be capable of binding to the base portion and to the second material. The second material may be capable of binding to the first material and to the surface layer. The substrate of the invention may comprise additional layers located between the surface layer and the base portion. Such additional layers may comprise one or more bilayers of surface layer material and binding layer material.

Further provided in accordance with the invention is a use of a substrate according to the invention for the inhibition of cell growth. The present invention has been shown to inhibit the growth of a number of cell types. The examples described later show inhibition of mammalian cells (fibroblast and epithelial cells), bacterial cells (Staphylococcus aureus, Psuedomonas aeruginosa and Streptococcus mutans), and fungal cells (Aspergillus niger, Candida albicans and Aureobasidium pullulans).

The topographical features are preferably provided by means of controlled deposition onto the base portion of a surface layer capable of adhering to the substrate. Such adherence may be chemical or physical.

Thus, in one of its aspects this invention relates to methods for tailoring the surface topography of substrates using the controlled deposition of thin films of nanoscale material onto an underlying base portion so as to inhibit cell growth on the treated surface. For example, the substrate may be used in the manufacture or treatment of a hygienic work surface (such as in a restaurant), the surface of a fluid conduit (such as a beer delivery tube or air conditioning duct), or even a medical implant (such as an intraocular lens).

The present invention further provides a method for manufacturing a substrate useful for the inhibition of cell growth comprising the steps of:

a) providing a base portion, a material suitable for forming a surface layer on the base portion, and a binding material suitable for forming a binding layer between the base portion and the surface layer;

b) contacting the base portion with the binding material under conditions effective for at least partial binding of the binding material to the base portion; and

c) contacting the at least partially bound binding material with the surface layer material under conditions effective for at least partially binding the surface layer to the binding material to form a surface layer at least partially covering the base portion, the surface layer comprising topographical features having at least one nanoscale dimension of from about 1 to about 200 nm.

If necessary, the method may further comprise the step of completing the binding of the binding material to the base portion and/or the surface layer.

Also provided in accordance with the invention is a method for manufacturing a substrate useful for inhibition of cell growth comprising the steps of:

a) providing a base portion, a material suitable for forming a surface layer on the base portion, and a binding material suitable for forming a binding layer between the base portion and the surface layer;

b) contacting the surface layer material with the binding material under conditions effective for at least partial binding of the binding material to the surface layer material; and

c) contacting the at least partially bound binding material with the base portion under conditions effective for at least partially binding the base portion to the binding material to form a surface layer at least partially covering the base portion, the surface layer comprising topographical features having at least one nanoscale dimension of from about 1 to about 200 nm.

If necessary, the method may further comprise the step of completing the binding of the binding material to the base portion and/or the surface layer.

Thus, in one of its aspects the invention provides a substrate, modified with a surface of deposited nanoscale material in order to control and inhibit the cellular response that occurs as a result of cell contact or interaction. Further provided is a process of tailoring surface topography by the deposition of nanoscale material onto an underlying substrate material so as to inhibit the cellular response that occurs as a result of cell contact or interaction with that surface.

In one preferred embodiment of the invention there is provided a cell growth inhibiting substrate comprising, a base portion being provided on a surface thereof, by means of controlled deposition onto the base portion, of a substance capable of adhering to the base portion, with topographical features having at least one nanoscale dimension and a cell or tissue growth inhibition region thereon and/or therebetween, the topographical features being deposited in a densely packed array with a separation between nearest neighbor similar topographical features of not more than 1000 nm.

In another preferred embodiment of the invention there is provided a method of manufacturing a substrate for the inhibition of cell growth comprising, providing a base portion, depositing onto the base portion a substance capable of adhering thereto in order to provide the substrate with topographical features having at least one nanoscale dimension the deposit being densely packed with separation between the topographical features of not more than 1000 nm, and providing the substrate with a cell or tissue growth inhibition region on and/or between the topographical features.

The base portion may be selected from any suitable material, depending for example on whether it is intended to inhibit or control cell or tissue growth on the base portion material itself or only on the covering surface layer. The end use of the substrate may also help determine the choice of base portion material, a relatively rigid material being used in the manufacture hygienic work surfaces, for example. The base portion may comprise a single material or may comprise two or more layers of different materials. The base portion is preferably formed from a material selected from polymers, glasses, ceramics, carbon, metals, composites and paper (including tissue paper). The base portion may also comprise an existing surface that is modified in accordance with the present invention. This may for example be an existing work surface or swimming pool surface, to which a surface layer is applied.

The surface layer is preferably formed from a material selected from polymers, glasses, ceramics, carbon, metals and composites. Suitable surface layer materials include silica, gold and silver. The surface layer may comprise colloidal particles of these or other materials and such colloidal particles may be nano-particulate, for example having mean diameters of from about 5 to about 80 nm.

The binding layer may comprise one or more substances capable of adhering to the base portion and the surface layer material. Suitable binding layer materials include polymers, surface active agents, reactive chemical ligands and polycationic materials. Preferably the adhering substance is insoluble or sparingly soluble. Inorganic, organic, metallic and polymeric materials, or mixtures of one or more thereof, may be used.

The substrates, methods and uses of the invention have advantages over conventional engineered surfaces used in medical engineering and methods for making them. The growth of different cells can be inhibited by selecting different topographies, as cells respond differently to different physical environments. The invention therefore provides a valuable tool for use in medical engineering. The substrates of the invention may also be adapted to provide robust surfaces for use, for example, in hospitals, restaurants and kitchens where it is desirable to discourage eukaryotic and prokaryotic cell growth on surfaces such as bench tops, walls, floors and fluid delivery tubes.

The substrate may be applied to, or incorporated into a wide variety of products and surfaces. For example, the substrate may be used in the following list (which is by no means exhaustive) of products:
Sanitary ware—including toilets, baths, basins, shower enclosures and respective fittings;
Fluid conduits—including liquid delivery tubes for potable liquids and air conditioning ducts;
Filters—including potable liquid and air conditioning filters;
Food preparation and storage apparatus—including cutlery, chopping boards and storage containers;
Work surfaces—including kitchen and restaurant tables;
Wall and floor coverings—including tiles, sheeting, laminated surfaces, cladding, painted surfaces, wall paper and fabric coverings;
Surgical and medical apparatus—including surgical implements and devices, catheters, needles, percutaneous devices, stoma care products, operating theatre devices, beds, chairs, tables, bedding and gowns;
Medical dressings—including plasters, wound dressings and sticking tape;
Diapers—including diapers for babies, incontinence pads and pants;
Dentures—including denture plates, dental bridges and false teeth; and
Implants—including intraocular lenses.

It will be apparent to the skilled person, that the substrate of the invention can be manufactured using materials which are non-toxic to human health. For example, the use of amorphous colloidal silica (SiO₂— silicon dioxide) is known to be non-toxic to humans. Other non-toxic colloidal materials may also be employed, such as gold or silver.

The substrate may be applied to an existing product or surface by a number of methods, such as spraying or washing. These methods are particularly suited to products or surfaces having complex geometries (such as dentures plates) or surfaces which are inaccessible (such as the interior of beer delivery tubes).

If necessary, the substrate may be manufactured from materials which allow the substrate to be flexible so that it can be applied to, or integrated with products which are flexible or allow for a certain degree of flexibility.

### BRIEF DESCRIPTION OF THE SEVERAL VIEWS OF THE DRAWINGS

The invention will now be more particularly described with reference to the following Figures and Examples in which:

FIG. 1 shows an image of a first substrate in accordance with the invention, the image being generated by means of atomic force microscopy (AFM);

FIG. 2 shows an image generated by a light microscope of cells on a substrate in accordance with the invention;

FIG. 3 shows an image generated by a light microscope of cells on a substrate in accordance with the invention;

FIG. 4 shows an image generated by a light microscope of a further example of cells on a substrate in accordance with the invention as shown in FIG. 2;

FIG. 5 shows an image generated by a light microscope of a further example of cells on a substrate in accordance with the invention as shown in FIG. 3;

FIG. 6 shows an image generated by a light microscope of cells on the edge of a poorly adhered surface layer on a substrate in accordance with the invention;

FIG. 7 shows an image generated by a light microscope of cells on the edge of a poorly adhered surface layer on a substrate in accordance with the invention;

FIG. 8 shows a high magnification image generated by a light microscope of cells on the edge of a poorly adhered surface layer on a substrate in accordance with the invention;

FIG. 9 shows an image generated by a light microscope of cells on a substrate in accordance with the invention;

FIG. 10 shows an image generated by a light microscope of cells on a substrate in accordance with the invention;

FIG. 11 shows an image generated by a light microscope of cells on a control substrate following air plasma treatment;

FIG. 12 shows an image generated by a light microscope of cells on a substrate following air plasma treatment in accordance with the invention;

FIG. 13 shows an image generated by a light microscope of cells on a substrate incorporating gold particles in accordance with the invention;

FIG. 14 shows an image generated by a light microscope of S. aureus cells on a control glass substrate;

FIG. 15 shows an image generated by a light microscope of S. aureus cells on a substrate incorporating gold particles in accordance with the invention,

FIG. 16 shows digital images of mould growth on untreated, Zetag™ and 7, 14 and 21 nm silica coated ceramic tiles as described in Example 6;

FIG. 17 shows optical micrographs of the growth of A. niger on (a) control and (b) 14 nm silica treated glass microscope slides as described in Example 6. The arrows (→) point to: (a) representative hyphal growth forms on untreated glass and (b) round spores of A. niger on silica treated glass. All images were captured at ×400 magnification (Carl Zeiss light microscope);

FIG. 18 is a schematic representation of the nano-scale surface patterning procedure used in Example 6;

FIG. 19 shows digital images of surface associated growth of A. niger after 6 days humid incubation at 25° C., on glass microscope slides coated in 7 nm colloidal silica particles (7-1 to 7-5), with polymer only (P1 to P5) and uncoated glass controls (G-1 to G-5) as described in Example 6. Each image represents a random field viewed at x40 magnification with a Kyowa Medilux-12 light microscope and captured using a Nikon Coolpix-990 digital camera. While a 10 mm graticule with 1 mm divisions was used, scale bars are included;

FIG. 20 shows digital images of surface associated growth of A. niger after 6 days humid incubation at 25° C., on glass microscope slides coated in 14 nm colloidal silica particles (141 to 14-5), with polymer only (P1 to P5) and uncoated glass controls (G-1 to G-5) as described in Example 6. Each image represents a random field viewed at ×40 magnification with a Kyowa Medilux-12 light microscope and captured using a Nikon Coolpix-990 digital camera. While a 10 mm graticule with 1 mm divisions was used, scale bars are included;

FIG. 21 shows digital images of surface associated growth of A. niger after 6 days humid incubation at 25° C., on glass microscope slides coated with 21 nm colloidal silica particles (21-1 to 21-5), with polymer only (P1 to P5) and uncoated glass controls (G-1 to G-5) as described in Example 6. Each image represents a random view viewed at ×40 magnification with a Kyowa Medilux-12 light microscope and captured using a Nikon Coolpix-990 digital camera. While a 10 mm graticule with 1 mm divisions was used, scale bars are included;

FIG. 22 shows a schematic representation of a typical sample of a partially coated Petri dish with a silica coated portion and an untreated portion as described in more detail in Example 7. The effect of the nanoparticulate silica coated portion can be clearly monitored on the same sample dish;

FIG. 23 shows optical micrographs of C. albicans on (a) an untreated portion, (b) a partially coated portion and (c) a 7 nm silica coated portion of the Petri dish captured at ×100 magnification (Kyowa Medilux-12 light microscope) as described in Example 7;

FIG. 24 shows optical micrographs of A. pullulans on (a) untreated glass, (b) 7 nm silica coated slides and (c) Zetag™ coated glass surfaces (Carl Zeiss light microscope), as described in Example 8;

FIG. 25 shows the results of the experiments described in Example 9. In particular, the amount of colony forming units (CFUs) after 2 h microbial retention assays (n=3) after rinsing and vigorous stirring of 1 cm² PET substrates in sterile PBS are shown in respect of 1. Staphylococcus aureus, 2. Psuedomonas aeruginosa and 3. Streptococcus mutansas; and

FIG. 26 shows the results of the experiments described in Example 9. In particular, the amount of colony forming units (CFUs) after 24 h microbial retention assays (n=3) after rinsing and vigorous stirring of 1 cm² PET substrates in sterile PBS are shown in respect of 1. Staphylococcus aureus, 2. Psuedomonas aeruginosa and 3. Streptococcus mutansas.

### DETAILED DESCRIPTION OF THE INVENTION

Substrates in accordance with the present invention are prepared generally with a base portion, a binding layer, and a surface layer. The base portion provides support as well as a suitable surface area for the application of the binding layer and surface layer. Typically, the base portion of the novel substrate is selected with characteristics appropriate for the environmental conditions of which the substrate will be subjected and is generally formed from polymers, glasses, ceramics, carbons, metals, composites, papers, and cellulosic materials.

A preferred base portion for the substrate is polystyrene which is a polymer comprised of the monomer styrene (vinyl benzene) and advantageously can be melted and molded or extruded and then resolidified to form a desired shape for the base portion. Polystyrene is often utilized in common laboratory equipment comprising such articles as test tubes and Petri dishes, typically formed from injection molding. Advantageously, the substrate of the present invention may utilize polystyrene thus imparting novel properties on a material proven capable for a variety of research and diagnostic tasks. Yet furthermore, while polystyrene is a preferred base portion of the substrate of the present invention, the base portion may also comprise other polymers including other thermoplastics such as polyethylene, polypropylene, and nylon; thermosets such as vulcanized rubbers, bakelite, and resins; elastomers such as polybutadiene, polyisoprene, and other saturated and unsaturated rubbers; and other rigid or semi-rigid polymeric materials.

In further preferred embodiments of the substrate, the base portion may comprise ceramics and glass, ideally suited for sanitary ware, medical implants, work surfaces, as well as other uses wherein ceramics and glass are generally known to be utilized. More specifically, ceramics for the base portion may include white ware ceramics such as porcelain useful for comprising toilets, baths, basins, showers and respective fittings.

Addition base portions for the substrate including but not limited to metals, carbons, papers and cellulosic materials may be utilized, thus providing a variety of materials and compositions with desirable cellular response characteristics. With these materials as well as polymers and ceramics and glass, multiple types of materials may be combined in layers to comprise the base portion of the substrate.

The binding layer of the substrate of the present invention may comprise one or more materials capable of adhering to the base portion as well as providing a bonding surface for the surface layer. The binding layer may include but is not limited to polymers, surface active agents, reactive chemicals, ligands and polycationic materials. Preferably the binding layer is insoluble, substantially insoluble, or sparingly soluble. One selection of cationic materials useful for creating the binding layer of the substrate includes Zetag™ of Millienium Technologies Ltd. Further binding materials may include inorganic, organic, metallic and polymeric material and combinations thereof for binding the surface layer to the base portion of the novel substrate.

Yet furthermore, the binding layer may include one or more layers, and further may include a plurality of types of materials. The first layer of the binding layer may be capable of binding to the base portion and to the second layer of the binding layer whereas the second layer may be capable of binding to the first binding layer and to the surface layer. Additionally, additional binding layers may be included between the first and the second binding layers.

The surface layer comprises a nanoscale material which may impart cellular control characteristics to the novel substrate. Generally, the surface layer is selected from materials including polymers, glasses, ceramics, carbons, metals and composites and preferably includes silica, gold, silver or combinations thereof. The surface layer may comprise particles of these or other materials with an average diameter of from about 5 nanometers to about 80 nanometers thus forming a colloidal surface layer. Preferably amorphous colloidal silica may be utilized as it is known to be substantially non-toxic to humans.

In order to create the novel substrate, a suitable base portion is first selected based on the desired use and environmental condition to which the substrate will be subjected. Preferably, the base portion is washed with methanol and water prior to application of the binding layer.

The binding layer may be subsequently be applied to the base portion, in a variety of manners, including both immersion and spray-treating so as to fully apply the binding layer to the desired surface for the cellular control properties. Advantageously, spray-treating of the binding layer may be used in conjunction with base portions having complex geometries as well interior surfaces for which immersion would not be as efficient. Multiple layers applied through similar or different techniques may be applied to the base portion, depended on the chosen binding layer materials as well as eventual application of the substrate. One category of binding layers includes cationic polymers which may function as to ionically bind the particles of the surface layer to the binding layer of the substrate.

The surface layer may also be applied to the combination base portion and binding layer through either immersion, spray-treating, or through other application techniques known in the art for applying colloids. Generally, the colloid comprises of from about a 5% to about a 85% colloidal particle solution, and more preferably of from about a 20% to about a 40% colloidal particle solution. Furthermore, the colloid characteristics are determined by particle type and size as well as the desired application of the finished substrate. Preferably, a silica colloid may be utilized in conjunction with a cationic binding layer, which should result in the formation of a ionically bound monolayer.

A substrate formed from the aforementioned steps may initiate a distinct cellular response affecting the both the morphology and adhesion of cells to the surface of the substrate this limiting cellular proliferation. Cell adhesion may be precluded or limited in both eukaryotic and prokaryotic cell types. In order to further illustrate the principles and operation of the present invention, the following examples are provided. However, these examples should not be taken as limiting in any regard.

### EXAMPLE 1

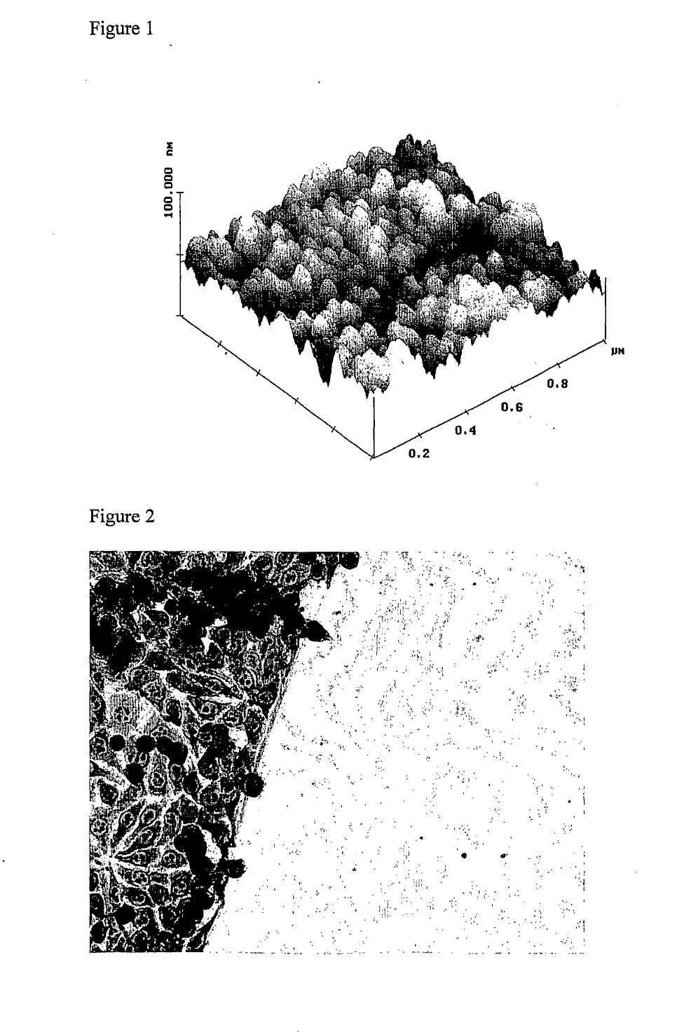

Three 10×10×1 mm² optically clear polystyrene (PS) squares are cut out from a plasma treated polystyrene culture dish. Each PS segment is then washed once with methanol followed by copious rinsing with deionized water (Millipore-Q 18.2 M). Each cleaned PS sample is then half immersed in an aqueous solution of 1 g L⁻¹ polycationic polymer (Zetag)™ for approximately 15 minutes to allow for the development of a monolayer of polycationic polymer on the PS surface. The polycationic derived PS samples are then removed from the polymer solution and washed copiously with deionized water (Millipore-Q 18.2 M) to remove excess polycationic polymer. The coated portions of each PS sample are then immersed in three different aqueous dispersions of silica solution (Ludox TM-50; HS-40 and SM-30, ex. Dupont de Nemours & Co.) containing approximately 40% w/w silica particles of approximately 21, 14 and 7 nm diameter respectively. A further aqueous dispersion of silica solution is also used which contains approximately 10% w/w silica particles of approximately 80 nm in diameter. The surfaces of each silica coated PS sample are then scanned using atomic force microscopy (AFM). An AFM image of the surface of a 21 nm coated sample is shown in FIG. 1, which shows a random close packed array of silica particles.

The samples of silica coated PS are then used as substrates in cell culture experiments. A suspension of clone L 929 mouse fibroblast established cell line is prepared from a culture maintained in Eagle's Minimum Essential medium with a 5% foetal calf serum supplement. The suspension is prepared at a cell concentration of approximately 1×10⁵ cell/ml. This is performed by immersing each PS sample in a cell culture medium containing established fibroblast cells for approximately 24 hours in an incubator at 37° C.

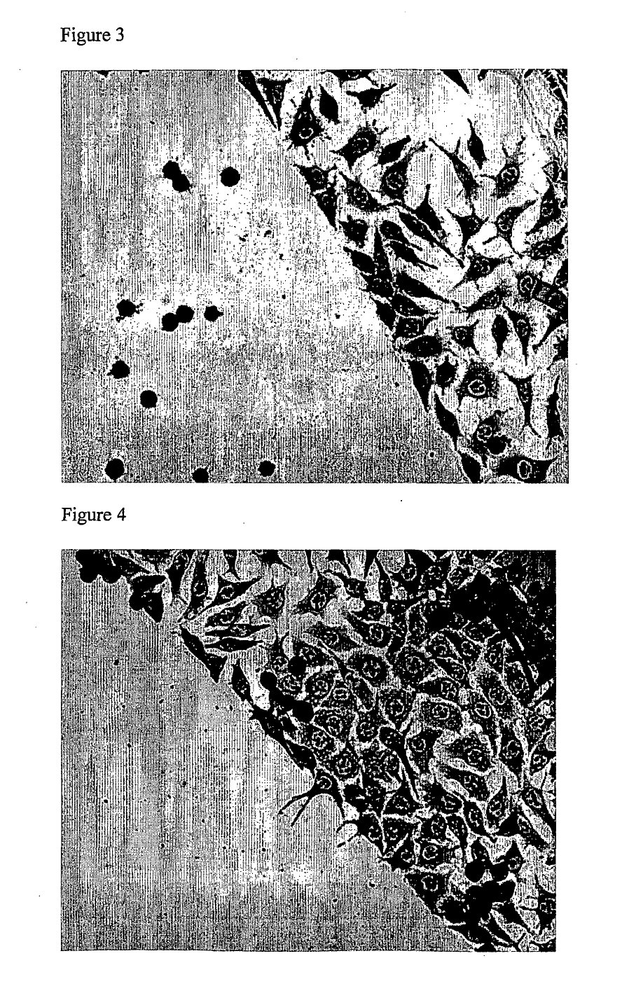

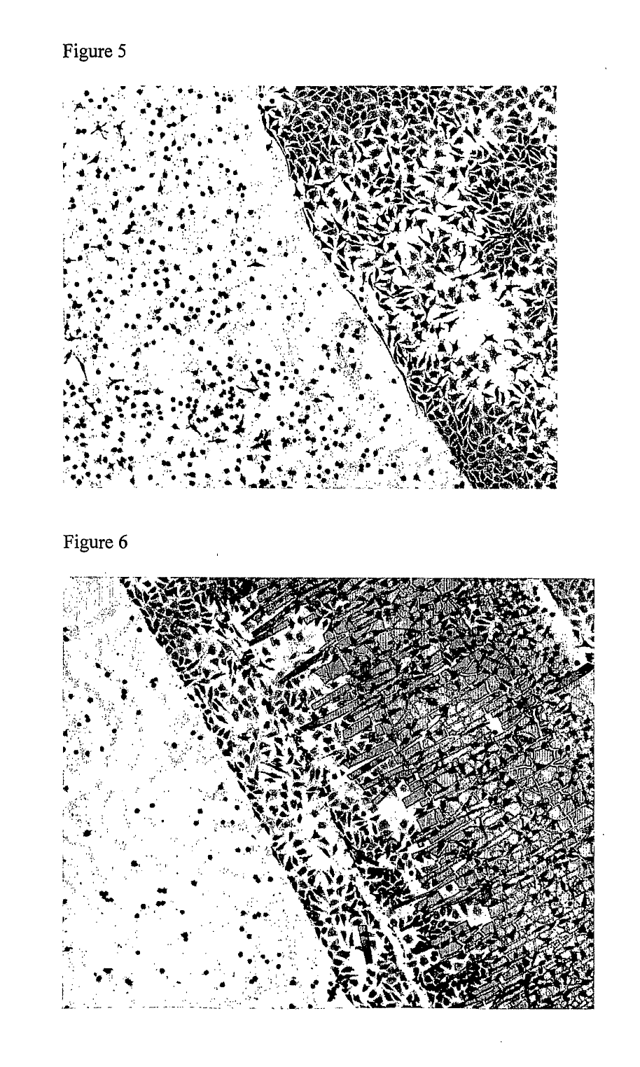

After this period the PS samples are removed from the culture medium and examined with an inverted phase-contrast light microscope. An image observed on 14 nm silica coated PS is shown in FIGS. 3 and 5. It can be seen that the cells develop as flat, extended entities on the surface of the clean PS indicating a strong affinity for the cells to attach and develop on the surface with a confluent morphology. This is in contrast to the treated segment of the PS culture dish where the cells remain spherical in solution and do not adhere to the silica coated PS surface.

In a variant of the previous experiment, the cell suspension is only applied to the untreated substrate and the cells are allowed to spread to the interface during a 48 hour incubation period. FIGS. 2 and 4 show the results of this experiment and clearly show the interface between the treated and untreated base substrates. The cells do not cross the interface. Adhesion is again inhibited on the nano-particulate coated substrate and cells on the untreated substrate have assumed a normal morphology.

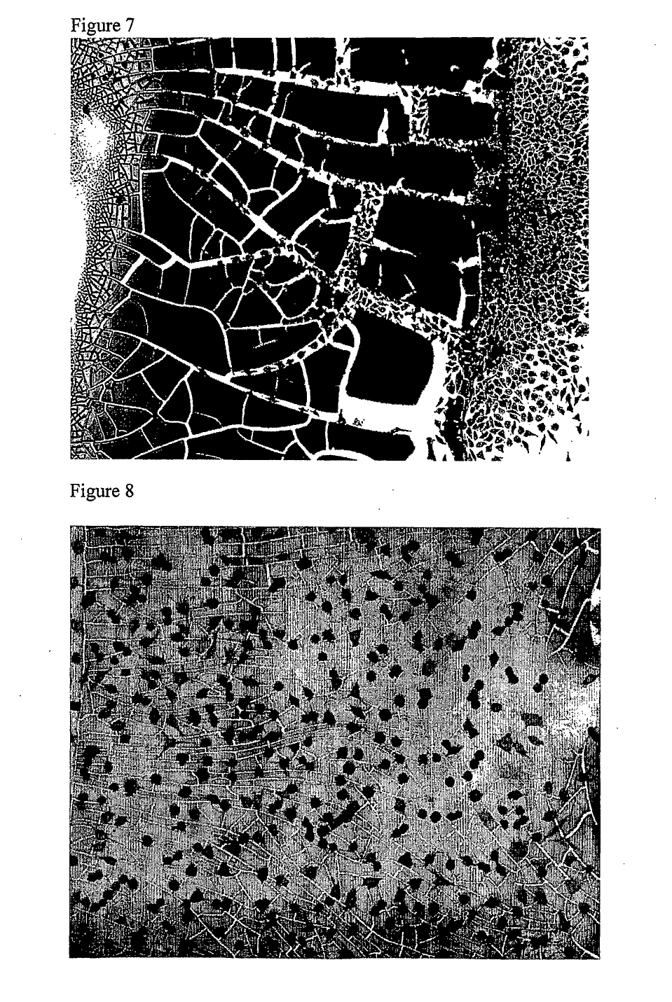

FIG. 6 shows the fibroblast cells at silica boundary. The silica boundary is clearly identified with silica coated surface. Cells on silica surface showed a rounded morphology. At the boundary there is a clearly identifiable dried/cracked silica layer, which is produced by a poorly adhered first layer of particles. Cells are showing a spread morphology on this surface, but appear to grow well in the voids in the silica and on the untreated surface. FIG. 8 is a higher magnification image of FIG. 6, and further illustrates the preference that the cells have for growth in the cracks within the silica. FIG. 7, is an image of a different part of the cracked silica and illustrates again the spread of fibroblast cells invading the cracks in the silica.

In a variant of the previous experiment, the cell suspension is only applied to the untreated substrate and the cells are allowed to spread to the interface during the 48 hour incubation period. As can be seen in FIG. 4, the cells do not cross the interface. Adhesion is again inhibited on the nano-particulate coated substrate and cells on the untreated substrate assume a normal morphology. Cells on the treated substrate retain a rounded morphology and are inhibited from spreading.

### EXAMPLE 2

In order to assess the growth characteristics of different cells on the substrate, bovine lens epithelial cells are applied to the glass substrate partially coated with nano-particulate material.

Primary bovine lens epithelial cells are obtained from the Unit of Opthalmology, The University of Liverpool at second or third passage and maintained in Dulbecco's Minimal essential Medium supplemented with 10% foetal calf serum. A cell suspension is prepared at a cell concentration of approximately 5×10⁴ cells/ml. 1 ml of this cell suspension is directly applied to both treated and untreated surface of a substrate prepared as described in Example 1. The cells are left in contact with the substrate for 30 minutes to allow cells adhesion, then the substrate is flooded with culture medium and maintained at 37° C./5% CO₂ for 48 hours. After this time the culture medium and non-adherent cells are removed. The substrate is then treated with 100% methanol in order to fix the cells and the substrate is stained with 0.04% methylene blue for 10 minutes.

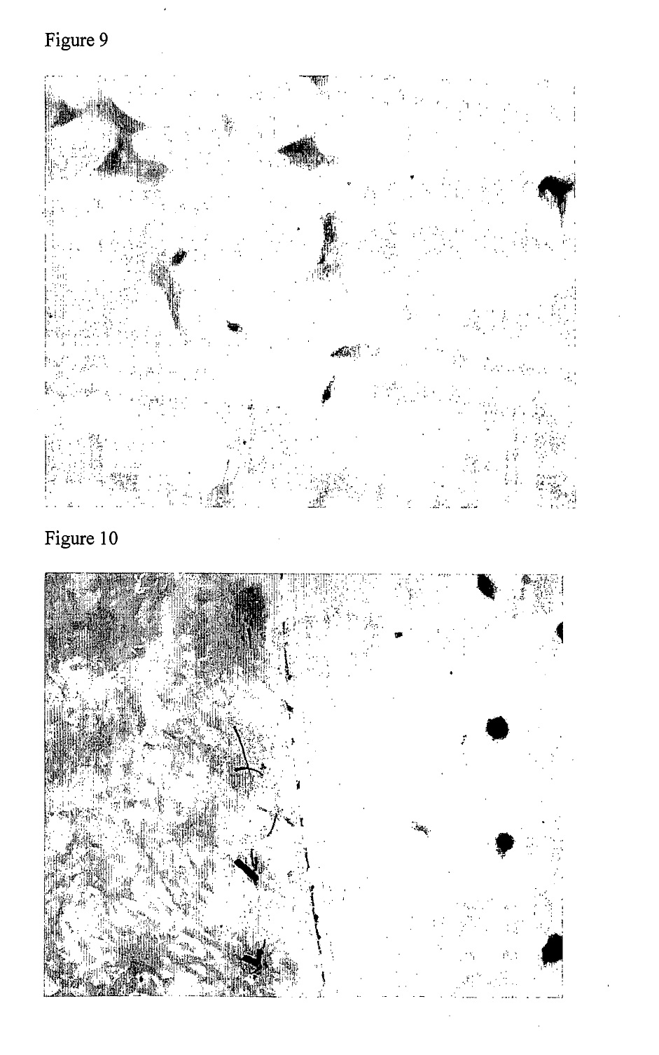

FIG. 9 shows an optical micrograph highlighting the interface between the treated and untreated base substrate. Cells on the treated substrate are fewer in number and have a changed morphology. There appears to be some inhibition of cell spreading. Furthermore, FIG. 10 which shows the results of an experiment that used primary bovine fibroblasts as opposed to epithelial cells as outlined above also highlights the interface between the treated and the untreated base substrate. Cells on the untreated substrate will assume a normal morphology, whilst cells on the treated substrate retain a rounded morphology and will remain in clumps and are inhibited from spreading.

In conclusion, epithelial cells are shown to behave in a similar manner to L 929 fibroblast cells on the treated and untreated substrate and it could also be postulated that other cell types will behave in a similar manner.

### EXAMPLE 3

PMMA and similar materials are often modified with an air glow discharge plasma in order to improve their wettability. Therefore an experiment will be conducted in order to assess the growth of fibroblast cells on a PMMA substrate which will be treated with an air plasma.

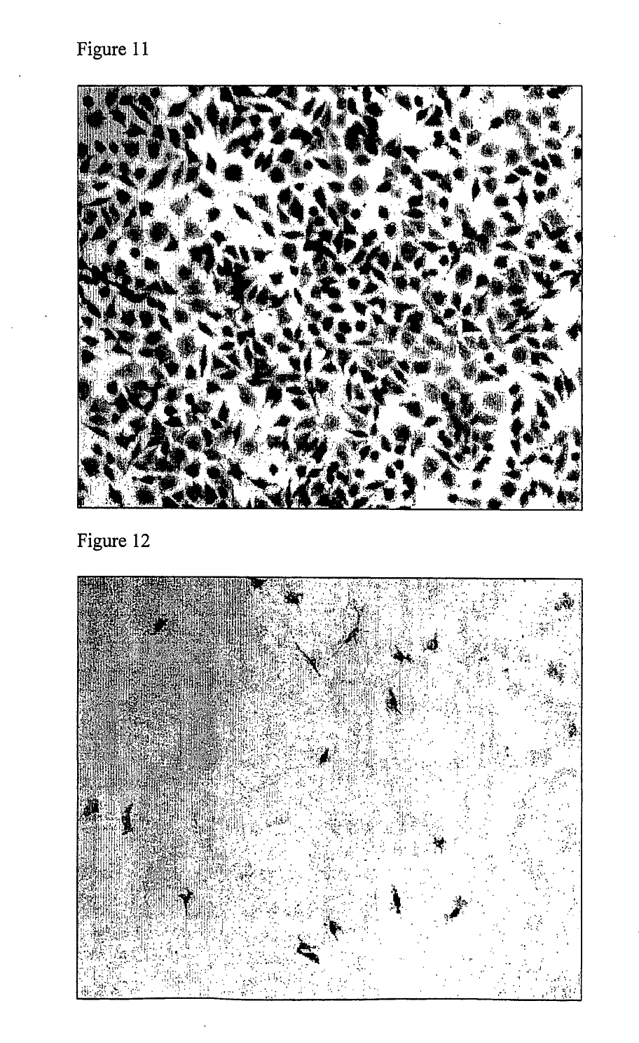

A suspension of clone L 929 mouse fibroblast established cell line is prepared from a culture maintained in Eagle's Minimum Essential medium with a 5% foetal calf serum supplement. The suspension is prepared at cell concentration of approximately 1×10⁵ cell/ml. 1 ml of the cell suspension is directly applied to the surface of an air plasma treated polymethylmethacrylate substrate which is prepared according to a standard protocol. The cells are left in contact with the substrate for 30 minutes to allow cells adhesion, then the substrate is flooded with culture medium and maintained at 37° C./5% CO₂ for 48 hours. After this time the culture medium and non-adherent cells are removed. The substrate is then treated with 100% methanol to fix the cells and the substrate is stained with 0.04% methylene blue for 10 minutes. FIG. 11 shows the results of this experiment and is an optical micrograph detailing normal confluent cell coverage and morphology on the substrate.

The L929 fibroblast cells are then tested on a PMMA substrate following air plasma treatment and subsequent nano-particulate coating.

The method is the same as outlined above and the cells are directly applied to the surface of an air plasma polymethylmethacrylate substrate as described in Example 1 with a subsequent nano-particulate coating. FIG. 12 shows the results of the experiment and is an optical micrograph demonstrating that the cell adhesion will be significantly inhibited when compared to the normal growth seen on FIG. 11.

### EXAMPLE 4

The cell growth modifying effects of a layer of silica nano-particles on PMMA, polystyrene and glass will be assessed in Examples 1 to 3. Further studies are directed to alternative nano-particles which could be used, one such material was gold.

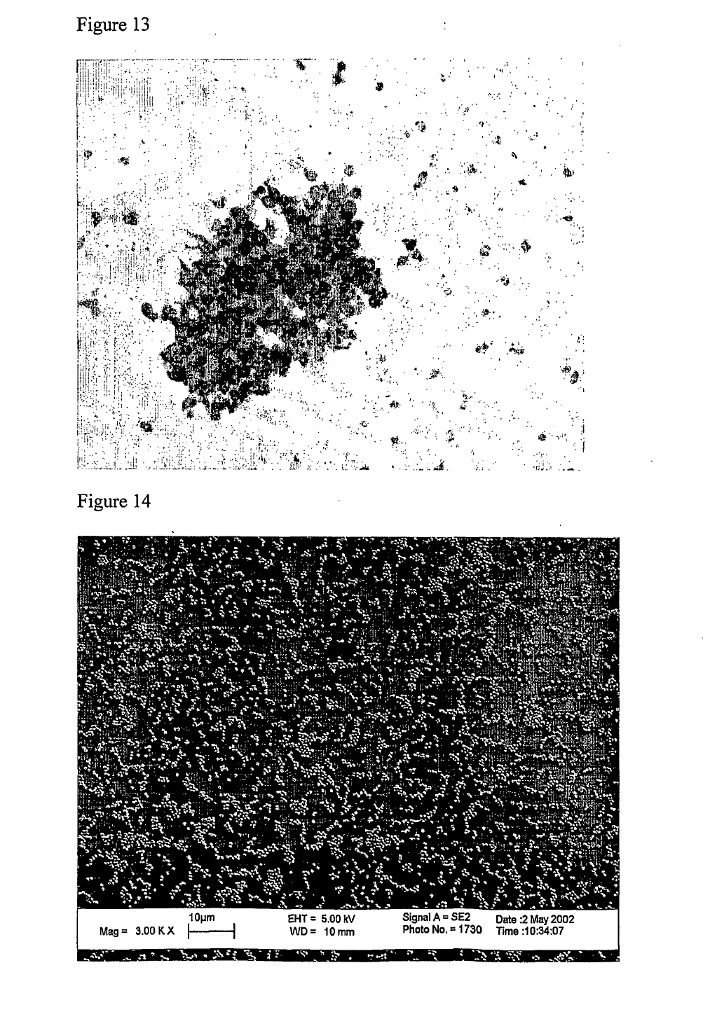

A gold particulate preparation of a 30 mM aqueous solution of hydrogen tetrachloroaurate (Aldrich) is mixed with 80 ml of a 50 mM solution of tetraoctyl ammonium bromide (Fluka) in toluene (AnalaR grade) forming a two phase mixture. The organic layer containing the [AuC₁₄]⁻ and [N(C₈H₁₇)₄]⁺ ions is then washed three times with de-ionized water. 25 ml of freshly prepared 0.4 M aqueous sodium borohydride (Fisons) is then added in small aliquots with vigorous stirring. The resulting colloidal gold solutions (approx. 5 nm in diameter) are deployed using the same procedure describe for the use of silica particles in Example 1. FIG. 13 shows the results of the experiment in an optical micrograph detailing following L929 contact with this substrate. The fibroblast cells display an abnormal morphology and there is evidence of cell lysis on the Au organosol nano-particulate substrate.

### EXAMPLE 5

Additional experiments are directed to non-mammalian cells. Staphylococcus aureus is chosen to investigate whether the nano-particle substrate could alter growth characteristics of prokaryotic cells.

A strain of S. aureus bacteria is prepared in full growth broth then placed directly in contact with a plain glass base substrate. A 1 ml suspension of the bacteria is presented to the substrate at a concentration of approximately 10⁷ cells/ml The bacterial will remain in contact with the substrate for 60 minutes. The substrate is then washed with distilled water and prepared for scanning electron microscopy by fixing in 2.5% gluteraldehyde and dehydrating in a series of alcohols.

FIG. 14 shows a scanning electron micrograph [×3000] which details the population of bacteria remaining on the substrate and it provides a quantification of S. aureus cells after 60 minutes of incubation on plain glass.

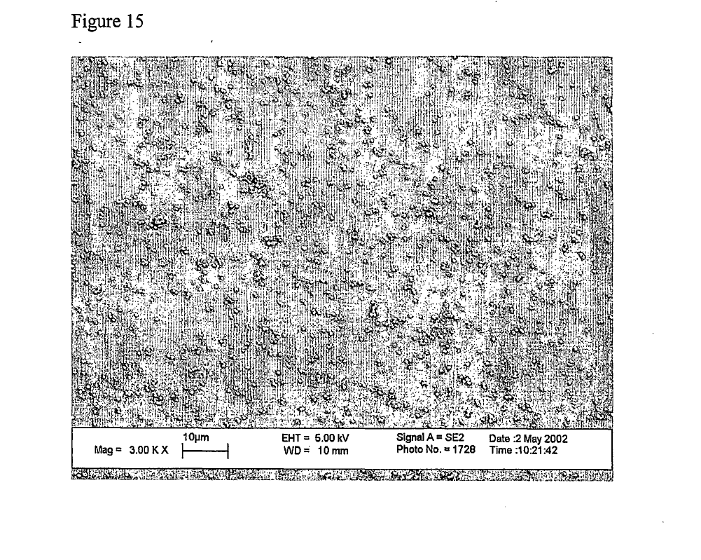

A further experiment is conducted that will utilize the same strain of S. aureus cells and will be provided to a slide by the same procedure as outlined above. The S. aureus will be presented to plain glass substrate coated with Au organosol which will be prepared in accordance to the protocol outlined in Example 4. FIG. 15 shows a scanning electron micrograph [×3000] which details the population of bacteria remaining on the substrate after 60 minutes of incubation. There is quantifiably lesser number of bacteria associated with the nanoparticulate substrate, which suggests that the nanoparticulate substrate inhibits the growth of S. aureus.

### EXAMPLE 6

An experiment is conducted to assess whether treating ceramic tiles and glass with colloidal silica will inhibit the growth of Aspergillus niger.

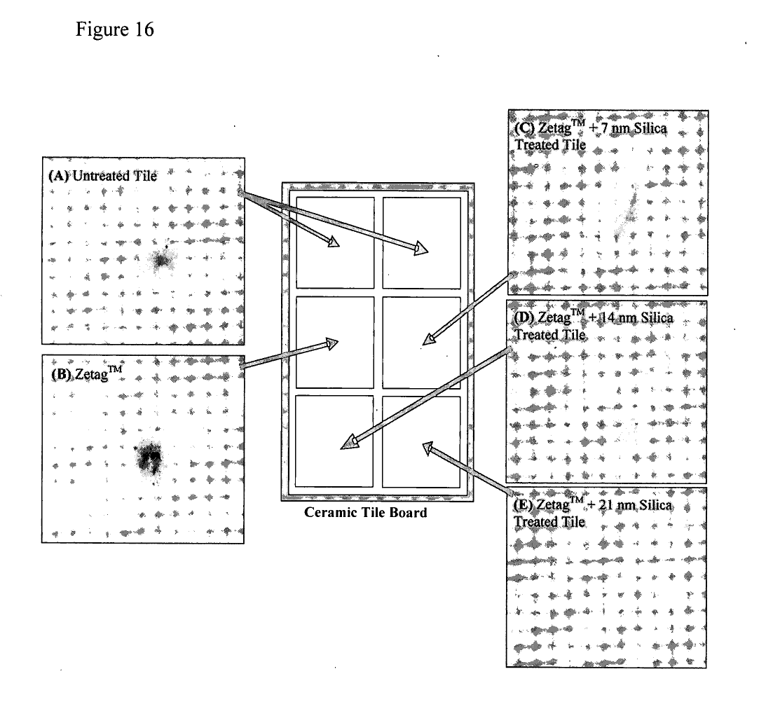

A tile board is treated with glacial acetic acid to simulate aging of the grout and tile surface then treated with the various coatings of Zetag™, Zetag™ plus 7, 14 or 21 nm silica or untreated ceramics as shown in FIG. 16. The surfaces are then seeded with A. niger as follows: A. niger is cultured on potato dextrose agar and spore suspensions prepared with potato dextrose broth. 100 μl of the suspension is transferred on to untreated and silica treated ceramic surfaces and incubated at 25° C. in a humidified incubator for a period of up to 14 days. Macro images from the ceramic tiles are captured digitally and are presented in FIG. 16.

After 7 days, fungal growth is evident with dark staining on the ceramic tiles, which highlighted the growth of fungal spores on untreated and Zetag™ coated tiles. On the silica coated tiles, it is noted that fungal growth is significantly less or absent. Less dark staining is apparent on 14 and 21 nm silica coated tiles and hence less spores adhere to the surface.

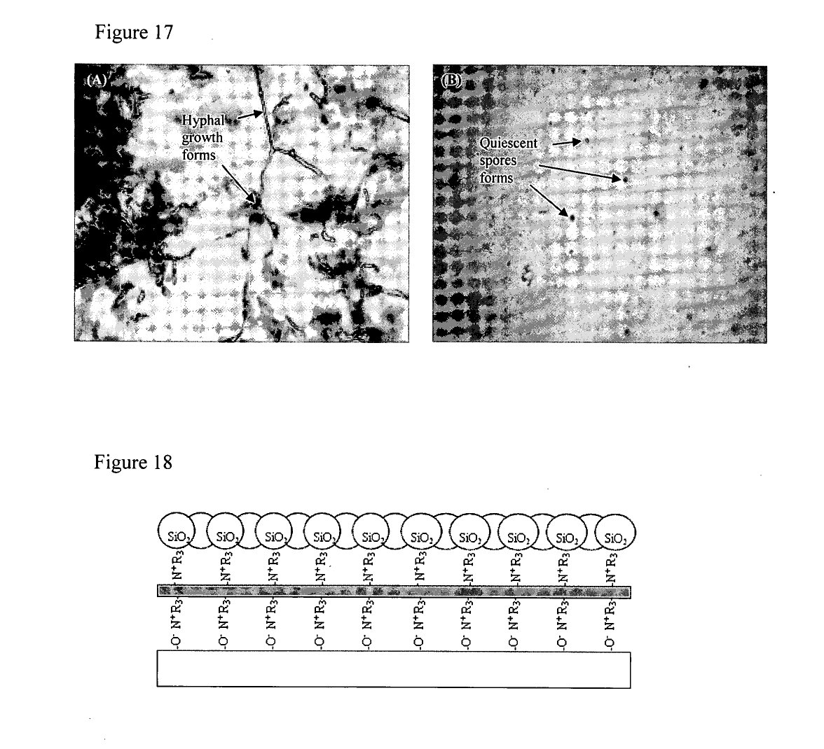

In FIG. 17(A), microscopic examination of fungal growth on the untreated control surface demonstrate production of hyphal structures and spores indicating a strongly adhered mould colony, whereas, on 14 nm silica coated surface as shown in FIG. 17(B) only quiescent spores are evident with no hyphal growth on the ceramic surface.

Parallel studies on glass are carried out to permit microscopic examination of fungal growth. Using glass coverslips coated with a commercial cationic polymer (Zetag™), anionic colloidal silica particles of sizes 7, 14 and 21 nm are deposited by dipping the slides into aqueous silica solutions. FIG. 18 schematically illustrates the different coating layers, and ionic interactions between the silica particles (shown as circles), polymer and glass surface (shown as an un-shaded rectangle). Adsorption of Zetag™ cationic polymer to the glass surface is achieved through (O⁻—N⁺R₃) ion pairs. Anionic silica nanoparticles adsorb on to the —N⁺R₃ groups from the polymer coating the glass surface. Spore suspensions are transferred on to untreated and silica treated glass microscope slides following the same procedures and growth conditions as described previously.

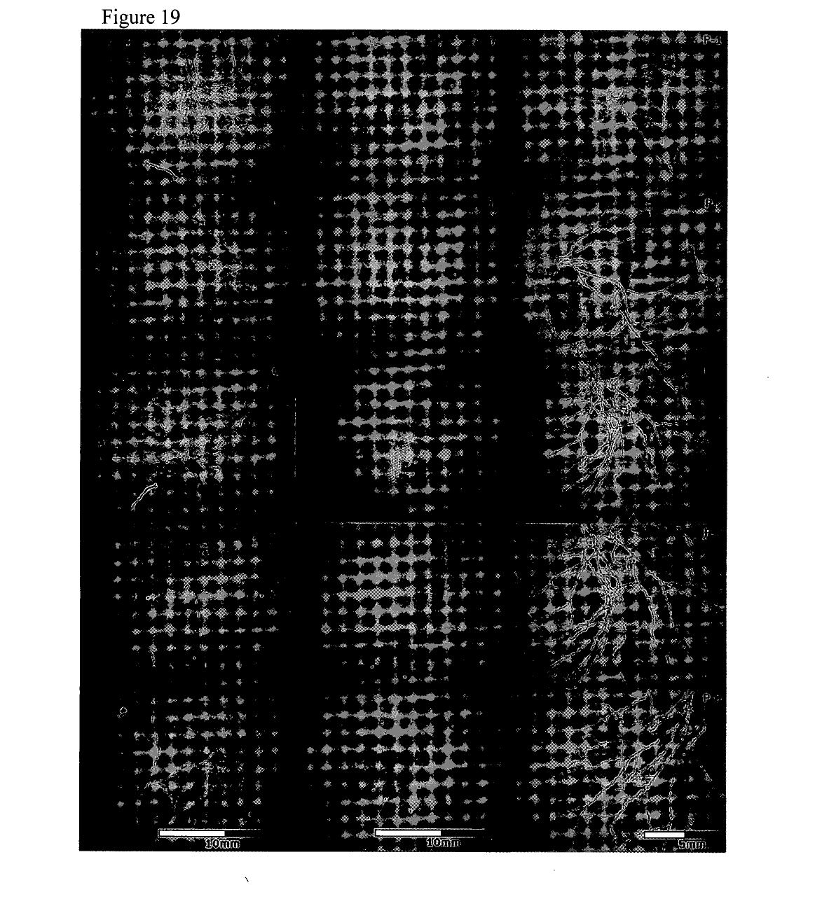

FIG. 19 shows the complete inhibition of fungal development in images 7-1 to 7-5, when using 7 nm silica coated glass microscope slides as a substrate. This contrasts with the confluent mycelial growth across the plain glass controls (FIG. 19 images G-1 to G-5), and the greater coverage and density found on the polymer treated slides (FIG. 19 images P-1 to P-5). While both control treatments show hyaline septate hyphae, the images of the polymer coated glass slides do show higher numbers of septae along each hypha when compared to those on the plain glass controls.

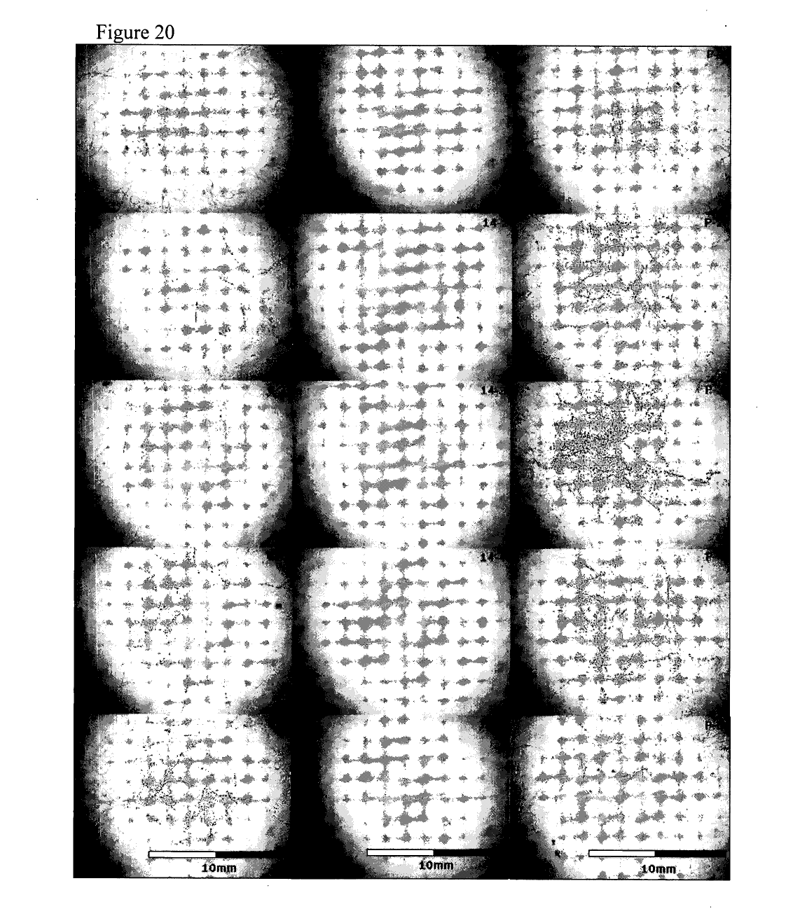

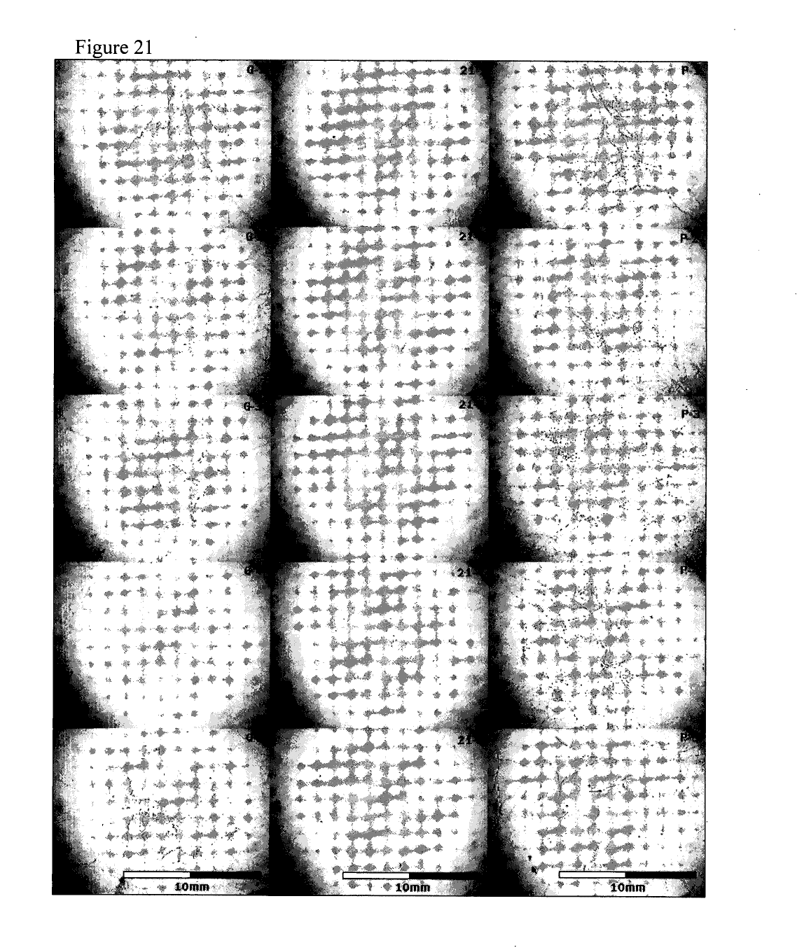

FIGS. 20 and 21 also present inhibitions of A. niger spore germination and surface colonization with the 14 nm and 21 nm silica coated glass slides. With the 14 nm (FIG. 20 images 14-1 to 14-5) and 21 nm (FIG. 21 images 21-1 to 21-5) showing similar developmental constraints to those seen with the 7 nm silica coatings (FIG. 19 images 7-1 to 7-5). A considerable amount of mycelial growth is present on both the plain glass and polymer only treatments in FIG. 20 (glass control images G-1 to G-5 and polymer control P-1 to P-5) and in FIG. 21 (glass control images G-1 to G-5 and polymer control P-1 to P-5). As with the two control groups in FIG. 19, there are differences in the number of hyphal septate between the two control treatments in FIGS. 20 and 21.

### EXAMPLE 7

An experiment is conducted to assess whether treating a Petri dish with silica will inhibit the growth of Candida albicans.

C. albicans adhesion and surface associated growth is observed on tissue culture polystyrene (TCPS) Petri dishes (90 mm diameter) which will be partially coated with 7, 14 or 21 nm silica exposing and untreated portion of the same dish. The lid of the Petri dish is removed and placed underneath it providing a sloping surface so that the dish could be partially coated with Zetag™ and a monolayer of silica. Partial coating of the Petri dishes is carried out using a two step procedure as follows:

1. 15 ml of Zetag™ (3.2 g/L) is placed into the dish to partially coat the surface and is left for 10 minutes before being removed, rinsed with water and allowed to air dry; and

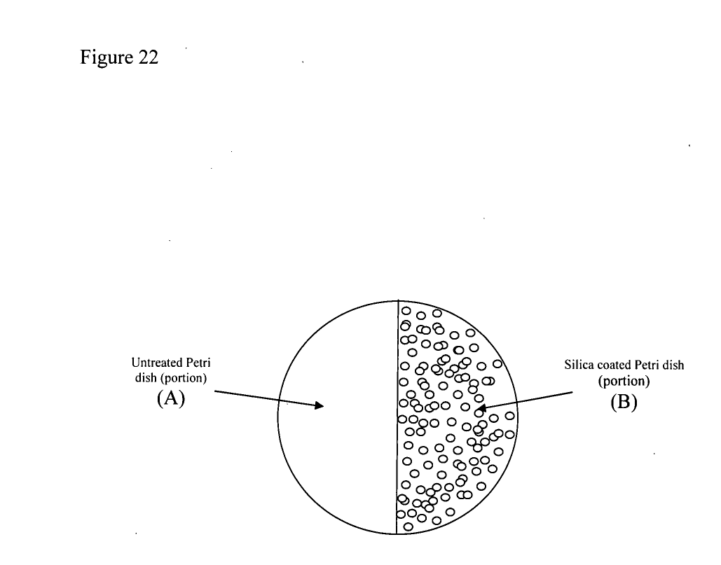

2. After drying, the Petri dishes are then partially immersed in 15 ml of silica (7, 14 or 21 nm diameter) before being further rinsed and allowed to air dry (FIG. 22 is shows a schematic drawing of a Petri dish which is half coated in silica).

C. albicans is maintained, grown and subcultured on Potato dextrose agar and stored at 4° C. Single colonies of C. albicans are propagated overnight while shaking at 37° C. in 10 ml of yeast peptone glucose (YPG) media (2% w/v D-glucose, 1% w/v yeast extract, 1% w/v malt extract and 0.1% w/v bacterial peptone). The overnight culture is diluted to an optical density (OD) of 0.1 at 550 nm in sterile YPG containing 5×10⁷ cell/ml. Horse serum is added to the YPG/cell suspension (10% v/v) to encourage hyphal development. 10 ml of the C. albicans culture is transferred aseptically on to partially coated Petri dishes and cultured for 24 h at 37° C. The Petri dishes are rinsed with 10 ml of sterile phosphate buffered silane (PBS) before being fixed with 4% (w/v) glutaraldehyde and stained with 0.4% (w/w) crystal violet.

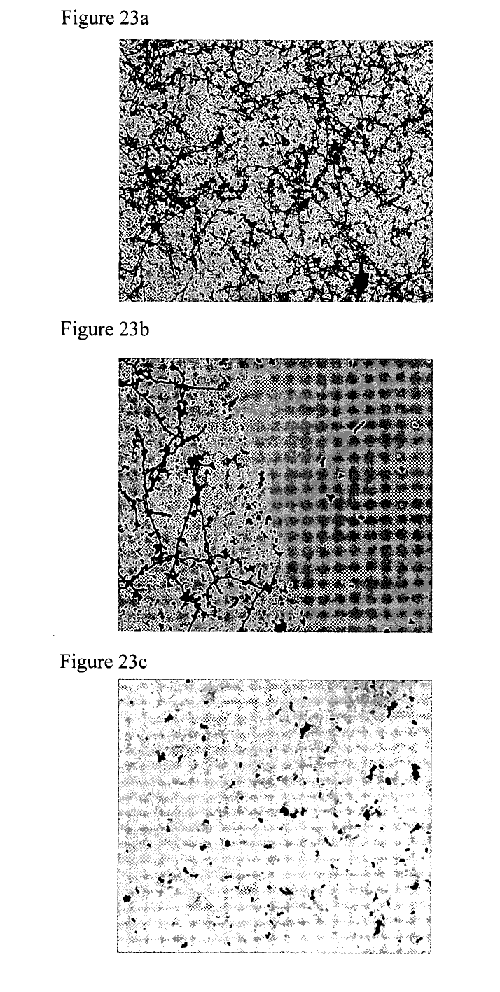

After 24 h in culture, the untreated portion of the dish will show a mixture of two distinct cell types. A high density of rounded budding yeast cells is apparent and highly branched cells, which represent hyphal development (FIG. 23a). This type of cell response is observed on Zetag™ coated substrates. On the portion of the dish which has been partially coated with 7 nm silica there is an obvious decrease in number and density of budding yeast cells and decreased cell attachment on these surfaces, which are readily removed upon rinsing with sterile PBS (FIG. 23b). There is no hyphal development and branching of cells on the silica coated portion of the plate (FIG. 23c).

Candida induced stomatitis is a considerable problem for immunocompromised denture wearers. Stomatitis is normally treated by antifungal drugs. However, the development of a Candida infection may be prevented by applying a coating similar to the one described above and this would hopefully negate the need for drug therapy.

### EXAMPLE 8

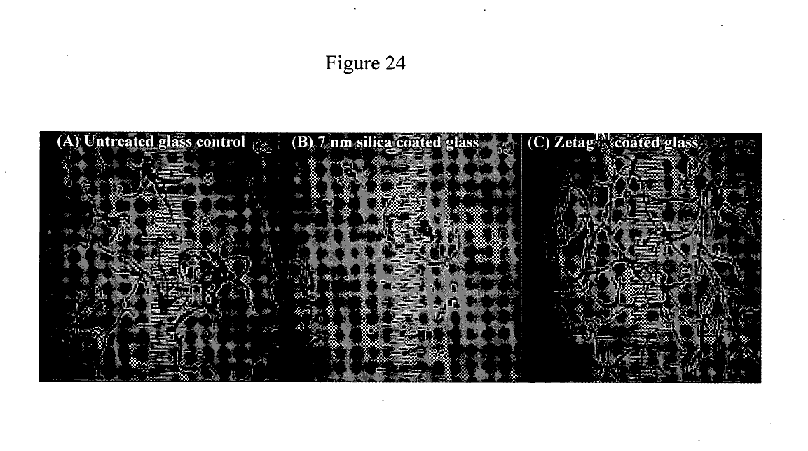

A further fungal species Aureobasidium pullulans is used to investigate surface growth after 3 days in humid incubation at 25° C. on glass microscope slides coated with 7 nm silica (FIG. 24b), with Zetag™ only (FIG. 24c) and untreated glass controls (FIG. 24a). Each image is captured from a random field of view at ×100 magnification with a Kyowa Medilux-12 light microscope and captured using a Nikon Coolpix-990 digital camera. A 10 mm graticule is used with 1 mm gradations between each major division and can be seen in the images in FIG. 24. Glass microscope slides are cleaned with 100% ethanol and are coated using the same two step procedure highlighted in Example 7. The surfaces are seeded with A. pullulans as follows: A. pullulans are cultured on potato dextrose agar slants and spore suspensions are collected and prepared in 0.5% (w/v) glycerol solution. The spore suspensions (108 spores/ml) were transferred directly on to each surface in 250 μl aliquots and incubated in a humidified container at 25° C.

Untreated glass control surfaces (FIG. 24a) show extensive hyphal development. Optical images of Zetag™ coated surfaces (FIG. 24c) show similar levels of hyphal formation and growth when compared with glass controls, however there is a decrease in the amount of spore forming structures, which gives rise to a smoother surface morphology. The 7 nm silica coated samples (FIG. 24c) give rise to markedly different patterns of reduced growth and development of A. pullulans, when compared to both control surfaces. The 7 nm silica coated surfaces have much smaller colonies with very little germ tube formation and with limited or no hyphal development.

### EXAMPLE 9

An experiment is conducted in order to investigate surface modification using nanoparticulate silica to reduce biofouling of surfaces.

Three bacterial species are used to test nanoparticulate silica coated surfaces, firstly by rinsing the surfaces to demonstrate their ease of removal and secondly, to monitor the effect of nanoparticulate silica coated substrates upon colonization and subsequent growth using the following test microorganisms: Staphylococcus aureus, Psuedomonas aeruginosa and Streptococcus mutans.

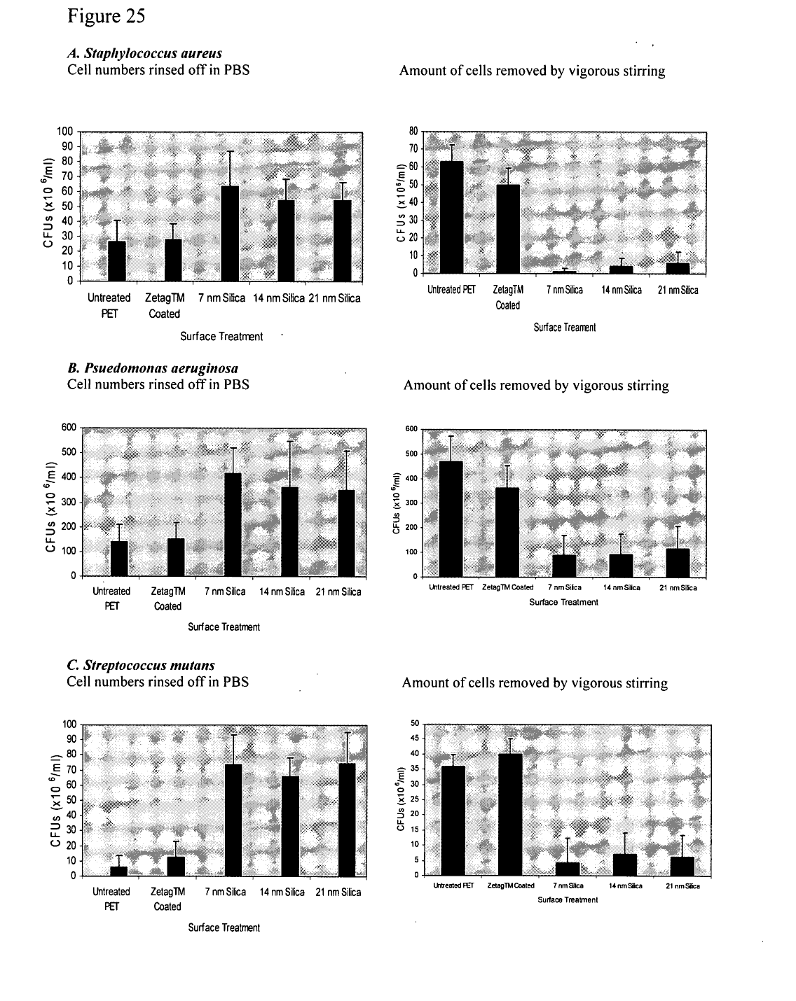

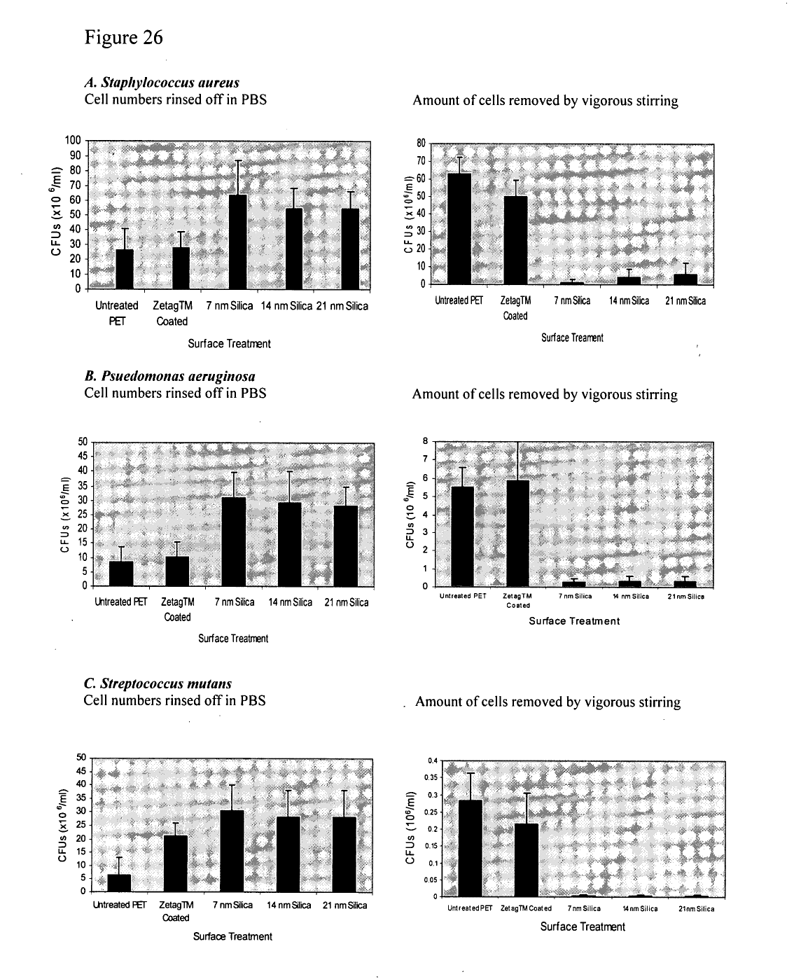

Microbial retention assays are carried out on untreated, Zetag™ treated and silica coated 1 cm² polyethylene tetrapthalate (PET) substrates following methods of Hirota and co-workers (Hirota, et al., FEMS Microbiology Letters. 248, 37-45, 2005). Overnight cultures of S. aureus and S. mutans will be prepared using 10 ml of bovine heart infusion (BHI) containing 1% glucose, and for P. aeruginosa 10 ml of tryptone soya broth (TSB) containing 1% glucose. Cell cultures are centrifuged and washed three times in 10 ml PBS to obtain cell suspensions for seeding. Cell suspensions of 0.5 ml of S. aureus, P. aeruginosa and S. mutans (1×10⁹ cells/ml) are placed in to 24-well plates containing control, Zetag™ treated and 7, 14 or 21 nm silica coated PET substrates and incubated at 37° C. over a 2 h (FIG. 25, 1-3) and 24 h time period (FIG. 26, 1-3).

The cell suspension is removed from each PET sample as well as the plate and transferred into a fresh, sterile 24 well plate. Each sample is rinsed three times with 1 ml of sterile PBS, which is collected in a universal container for viable cell counting on BHI and TSB agar plates to calculate the number of viable cells (counting colony forming units—CFUs) removed during rinsing of each surface. Furthermore, untreated, Zetag™ treated and 7, 14 or 21 nm silica coated PET samples are transferred into a universal container are 3 ml of sterile PBS is added and vigorously vortexed and agitated on an eppendorf mixer for 30 sec. The remaining supernatant is used for viable cell counting (colony forming units—CFUs) to estimate cell numbers adhering to each surface.

In FIG. 25, the amount of S. aureus removed by rinsing silica coated substrates will show a three fold increase in removal of cells when compared with controls. Few cells remain associated with silica coated samples after vortexing with log scale difference in cell numbers. The same cell response is evident when P. aeruginosa (FIG. 25, example 2) is used as the test species. Although, after vortexing a six fold difference in cell number (colony forming units) is evident. A similar result for S. mutans is obtained with almost a 70% removal of bacteria upon rinsing (FIG. 25, example 3). Similarly, fewer cells will remain attached to silica coated surfaces after vortexing with log scale difference in cell number. After 24 h (FIG. 26, examples 1-3), there is an overall ten fold reduction in retention of S. aureus, P. aeruginosa and S. mutans on viable cell counting due to a lack of available nutrients in the culture procedure. Even though this effect on nutrient availability is apparent, exactly the same reductions and trends upon rinsing surfaces and log scale difference in cell number when vortexing is apparent. It can be seen that microbial cells are readily removed upon rinsing and with agitation and that as a result, fewer or limited numbers of bacteria will remain adhered to silica coated surfaces when compared with controls. The bacteria will be weakly adhered to silica coated surfaces.

Advantageously, a substrate having a surface profile with nanometer scale dimensions is provided and created with relatively lesser costs than etched or ablated materials. The novel substrate may initiate a distinctive cellular response affecting the morphology, adhesion, and proliferation of cellular material not hereforeto seen.

The disclosures of all cited patents and publications referred to in this application are incorporated herein by reference.

The above description is intended to enable the person skilled in the art to practice the invention. It is not intended to detail all of the possible variations and modifications that will become apparent to the skilled worker upon reading the description. It is intended, however, that all such modifications and variations be included within the scope of the invention that is defined by the following claims. The claims are intended to cover the indicated elements and steps in any arrangement or sequence that is effective to meet the objectives intended for the invention, unless the context specifically indicates the contrary.

What is claimed is:

1. A cell growth inhibiting substrate comprising:
a base portion;
a surface layer covering at least part of the base portion, the surface layer providing the substrate with topographical features having at least one nanoscale dimension of from about 1 to about 200 nm; and
a binding layer between the base portion and the surface layer for binding the surface layer in place
wherein cellular proliferation on the substrate is limited.

2. The substrate according to claim 1, wherein the material of the binding layer is different from one or both of the materials of the base portion and the surface layer.

3. The substrate according to claim 1, wherein the surface layer comprises colloidal particles.

4. The substrate according to claim 3, wherein the colloidal particles comprise nano-particulate material.

5. The substrate according to claim 3, wherein the colloidal particles are from about 5 nm to about 80 nm in diameter.

6. The substrate according to claim 1, wherein the base portion is formed from a material selected from the group consisting of polymers, glasses, ceramics, carbon, paper, metals, composites and combinations thereof.

7. The substrate according to claim 1 wherein the base portion comprises polymer.

8. The substrate according to claim 1 wherein the base portion comprises glass.

9. The substrate according to claim 1 wherein the base portion comprises ceramic.

10. The substrate according to claim 1, wherein the surface layer is formed from a material selected from the group consisting of polymers, glasses, ceramics, carbon, metals, composites and combinations thereof.

11. The substrate according to claim 1, wherein the surface layer is formed from a material selected from the group consisting of silica, gold, silver and combinations thereof.

12. The substrate according to claim 1, wherein said topographical features have at least one nanoscale dimension of from about 1 to about 100 nm.

13. The substrate according to claim 1, wherein the surface layer comprises a single layer.

14. The substrate according to claim 1, wherein the binding layer comprises an organic material.

15. The substrate according to claim 1, wherein the binding layer is formed from a material selected from the group consisting of surface active agents, reactive chemical ligands, polycationic materials and combinations thereof.

16. The substrate according to claim 1, wherein additional layers are located between the binding layer and the surface layer.

17. The substrate according to claim 16, wherein the additional layers comprise one or more bilayers of surface layer material and binding layer material.

18. The substrate according to claim 1, wherein the substrate is flexible.

19. The substrate according to claim 1 wherein cell growth is inhibited.

20. The substrate according to claim 19 wherein the cell growth inhibited comprises cells selected from the group consisting of mammalian cells, bacterial cells, fungal cells and combinations thereof.

21. The substrate according to claim 19, wherein the substrate further comprises a product selected from the group consisting of sanitary ware, fluid conduits, filters, food preparation and storage apparatus, work surfaces, wall and floor coverings, surgical and medical apparatus, medical dressings, diapers, dentures and implants.

22. The substrate of claim 1 further comprising the substrate used in a hygienic work surface.

23. The substrate of claim 1 further comprising the substrate in a surface of a fluid conduit.

24. The substrate of claim 1 further comprising the substrate used in a surface of an implant.

25. The substrate of claim 1 further comprising the substrate used in an intraocular lens.

26. The substrate of claim 1 further comprising the substrate used in a surface of a denture.

27. A method for manufacturing a substrate for the modification of surface topography for the inhibition of cell growth comprising the steps of:
a) providing a base portion, a material suitable for forming a surface layer on the base portion, and a binding material suitable for forming a binding layer between the base portion and the surface layer;
b) contacting the base portion with the binding material under conditions effective for at least partial binding of the binding material to the base portion; and
c) contacting the at least partially bound binding material with the surface layer material under conditions effective for at least partially binding the surface layer to the binding material to form a surface layer at least partially covering the base portion, the surface layer comprising topographical features having at least one nanoscale dimension of from about 1 to about 200 nm.

28. The method for according to claim 27, wherein the method further comprises the step:
d) completing binding of the binding material to the base portion and/or the surface layer.

29. The method according to claim 27, wherein the material of the binding layer is different from one or both of the materials of the base portion and the surface layer.

30. The method according to claim 27, wherein the surface layer comprises colloidal particles.

31. The method according to claim 30, wherein the colloidal particles comprise nano-particulate material.

32. The method according to claim 30, wherein the colloidal particles are from about 5 nm to about 80 nm in diameter.

33. The method according to claim 27, wherein the base portion is formed from a material selected from the group consisting of polymers, glasses, ceramics, carbon, paper, metals, composites and combinations thereof.

34. The method according to claim 27, wherein the surface layer is formed from a material selected from the group consisting of polymers, glasses, ceramics, carbon, metals, composites and combinations thereof.

35. The method according to claim 27, wherein the surface layer is formed from a material selected from the group of silica, gold, silver and combinations thereof.

36. The method according to claim 27, wherein said topographical features have at least one nanoscale dimension of from about 1 to about 100 nm.

37. The method according to claim 27, wherein the surface layer comprises a single layer.

38. The method according to claim 27, wherein the binding layer comprises an organic material.

39. The method according to claim 27, wherein the binding layer is formed from the group consisting of a material selected from surface active agents, reactive chemical ligands, polycationic materials and combinations thereof.

40. The method according to claim 27, wherein the method further comprises the step of applying a second layer of the binding material and surface layer to the substrate.

41. The method according to claim 27, wherein the substrate is flexible.

42. The method according to claim 27, wherein the substrate is applied to an existing surface and/or the base portion is an existing surface.

43. The method according to claim 42, wherein the existing surface is located on a product selected from the group consisting of sanitary ware, fluid conduits, filters, food preparation and storage apparatus, work surfaces, wall and floor coverings, surgical and medical apparatus, medical dressings, diapers, dentures and implants.

44. The method according to claim 42, wherein the existing surface comprises a hygienic work surface.

45. The method according to claim 42, wherein the existing surface comprises a fluid conduit.

46. The method according to claim 42, wherein the existing surface comprises an implant.

47. The method according to claim 42, wherein the existing surface comprises an intraocular lens.

48. The method according to claim 42, wherein the existing surface comprises a denture.

49. The method for manufacturing a substrate according to claim 1, comprising the steps of:
a) providing a base portion, a material suitable for forming a surface layer on the base portion, and a binding material suitable for forming a binding layer between the base portion and the surface layer;
b) contacting the surface layer material with the binding material under conditions effective for at least partial binding of the binding material to the surface layer material; and
c) contacting the at least partially bound binding material with the base portion under conditions effective for at least partially binding the base portion to the binding material to form a surface layer at least partially covering the base portion, the surface layer comprising topographical features having at least one nanoscale dimension of from about 1 to about 200 nm.

50. A method according to claim 49, wherein the method further comprises the step:
d) completing binding of the binding material to the base portion and/or the surface layer.

51. A method of limiting cellular proliferation comprising the steps of:
a) providing a base portion, a material suitable for forming a surface layer on the base portion, and a binding material suitable for forming a binding layer between the base portion and the surface layer;
b) contacting the surface layer material with the binding material under conditions effective for at least partial binding of the binding material to the surface layer material to form an at least partially bound binding material;
c) contacting the at least partially bound binding material with the base portion under conditions effective for at least partially binding the base portion to the binding material to form a nanotopgraphical surface layer at least partially covering the base portion, the nanotopographical surface layer comprising topographical features having at least one nanoscale dimension of from about 1 to about 200 nm;
d) limiting cellular proliferation on the nanotopographical surface layer of the substrate.

52. The method of claim 51 wherein step d) further comprises inhibiting cellular growth.

53. The method of claim 51 wherein the limiting cellular proliferation of step d) comprises limiting eukaryotic cellular proliferation.

54. The method of claim 53 wherein the limiting eukaryotic cellular proliferation comprises limiting mammalian cellular proliferation.

55. The method of claim 51 wherein the limiting cellular proliferation of step d) comprises limiting prokaryotic cellular proliferation.

56. The method of claim 55 wherein the limiting prokaryotic cellular proliferation comprises limiting bacterial cellular proliferation.

57. The method of claim 51 wherein the limiting cellular proliferation of step d) comprises limiting fungal cellular proliferation.
